(19) United States
(12) Patent Application Publication
(10) Pub. No.: US 2006/0166967 A1
(43) Pub. Date: Jul. 27, 2006

(54) Use of r-10-hydroxy-10,11-dihydro-carbamazepine in neuropathic pain

(76) Inventors: Alyson Fox, London (GB); Stuart Bevan, London (GB)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/545,410

(22) PCT Filed: Feb. 16, 2005
(86) PCT No.: PCT/EP04/01451

§ 371 Date: Sep. 12, 2005

(30) Foreign Application Priority Data
Feb. 17, 2003 (GB) 0303615.9

(51) Int. Cl. A61K 31/55

(52) U.S. Cl. 514/217

(57) ABSTRACT

The present invention relates to the use of a mixture of the enantiomers of a compound of Formula (I) or of pharmaceutically acceptable salts of said enantiomers consisting of at least 55% of the R-enantiomer, most preferably of at least 98% of the R-enantiomer, and not more than 45 % of the S-enantiomer, most preferably not more than 2% of the Senantiomer, for the manufacture of a pharmaceutical composition for the treatment of neuropathic pain; to a method for the treatment of neuropathic pain; and to a pharmaceutical composition comprising as active agent a mixture of the enantiomers of the compound of formula I or pharmaceutically acceptable salts of said enantiomers consisting of at least 55% of the R-enantiomer and not more than 45% of the S-enantiomer.

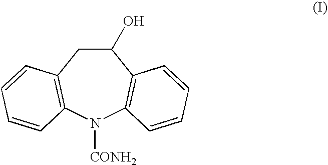

The present invention relates to new pharmaceutical uses of a carbamazepine derivative.

More particularly the present invention relates to new pharmaceutical uses for a mixture of the enantiomers of the carbamazepine derivative of formula I

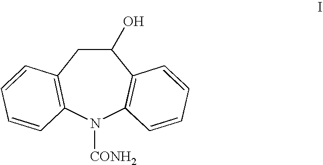

and its pharmaceutically acceptable salts.

Racemic MHD (formula I, 10-hydroxy-10,11-dihydro-carbamazepine), the main metabolite of the antiepileptic oxcarbazepine (Trileptal®), is well known from the literature [see for example Schuetz H. et al., Xenobiotica (GB), 16(8), 769-778 (1986)] and can be prepared synthetically starting from oxcarbazepine according to conventional methods. It was demonstrated that a racemate of the chiral carbamazepine derivative of formula I and both of its pure enantiomers show equal efficacy against epilepsy.

In accordance with the present invention, it was now surprisingly found that the R-enantiomer of the compound of formula I is substantially more efficacious than the S-enantiomer in the prevention and treatment of neuropathic pain.

Furthermore, it was surprisingly found that administration of the S-enantiomer at doses that reverses mechanical hyperalgesia is also associated with marked side-effects, principally ataxia and catalepsy, whereas comparatively mild side-effect are observed with the R-enantiomer at the tested doses.

Hence, the present invention pertains to the use of a mixture of the enantiomers of the compound of formula I or of pharmaceutically acceptable salts of said racemate consisting of at least 55% of the R-enantiomer and not more than 45% of the S-enantiomer, hereafter referred to as “the racemate”, for the treatment of neuropathic pain.

The term “neuropathic pain” as used herein includes, but is not restricted to, pain that frequently accompanies a range of different pathologies including nerve damage, amputation or conditions such as diabetes, post-herpetic neuralgia or trigeminal neuralgia. Preferably, the compounds of formula I can be employed for the treatment of diabetic neuropathic pain and post-herpetic neuralgia. The hyperalgesia and allodynia associated with neuropathic pain is particularly intractable and poorly treated in the clinic by treatments such as opiates or non-steroidal anti-inflammatory drugs.

The usefulness of the agents of the invention in the treatment of the above-mentioned disorders can be confirmed in suitable clinical studies as well as a range of standard tests including, e.g., the animal models described in the Examples below. The person skilled in the pertinent art is fully enabled to select a relevant test model to prove such usefulness. Suitable clinical studies are in particular randomized, double-blind, placebo-controlled, parallel studies in diabetic neuropathic pain patients.

For the treatment of neuropathic pain, appropriate dosage will of course vary depending upon, for example, the ratio of the different enantiomers, the host, the mode of administration and the nature and severity of the condition being treated. However, in general, satisfactory results in animals are indicated to be obtained at a daily dosage of from about 1 to about 300 mg of the racemate/kg animal body weight. In larger mammals, for example humans, an indicated daily dosage of the racemate is in the range from about 10 to about 3000 mg of a compound according to the invention, conveniently administered, for example, in divided doses up to four times a day.

The mixture may be administered in any usual manner, e.g. orally, for example in the form of tablets or capsules, or parenterally, for example in the form of injection solutions or suspensions.

The present invention also provides pharmaceutical compositions comprising a mixture of the enantiomers of the compound of formula I or pharmaceutically acceptable salts of said enantiomers consisting of at least 55% of the R-enantiomer and not more than 45% of the S-enantiomer in association with at least one pharmaceutical carrier or diluent for use in the treatment of neuropathic pain. Such compositions may be manufactured in a conventional manner.

Unit dosage forms may contain for example from about 2.5 mg to about 1000 mg of the racemate.

The invention further provides the use of a mixture of the enantiomers of the compound of formula I or of pharmaceutically acceptable salts of said enantiomers for the manufacture of a pharmaceutical composition for the treatment of neuropathic pain.

The invention further provides a method for the treatment of neuropathic pain in a subject in need of such treatment, which comprises administering to said subject a therapeutically effective amount of a racemate according to the invention.

Furthermore, the present invention provides a package comprising a pharmaceutical composition comprising a mixture of the enantiomers of the compound of formula I or a pharmaceutically acceptable salts of said racemate consisting of at least 55% of the R-enantiomer and not more than 45% of the S-enantiomer in association with at least one pharmaceutical carrier or diluent together with instructions for the use of said pharmaceutical composition in the treatment of neuropathic pain.

Preferably, the mixture consists of at least 85% of the R-enantiomer and not more than 15% of the S-enantiomer, more preferably of at least 98% of the R-enantiomer and not more than 2% of the S-enantiomer, most preferably of at least 99.5% of the R-enantiomer and not more than 0.5% of the S-enantiomer.

The mixtures of the invention can, e.g., be obtained by mixing the pure enantiomers of the compound of formula I. The pure enantiomers of the compound of formula I can be obtained by separation techniques starting from the racemate by procedures known as such. The racemate may be separated into its enantiomers through the formation of diastereomeric salts, for example by salt formation with an enantiomer-pure chiral acid, or by means of chromatography, for example by HPLC, using chromatographic substrates with chiral ligands.

In one embodiment of the invention, the pure enantiomers of the compound of formula I are prepared according to the procedures described in the Examples below.

The following Examples serve to illustrate the invention without limiting the invention in its scope.

### ABBREVIATIONS

Ac acetyl

aqu. Aqueous

dansyl 5-(dimethylamino)1-naphthalenesulfonyl

Et ethyl

HPLC high pressure liquid chromatography

Me methyl

NMR nuclear magnetic resonance

RT room temperature

THF tetrahydrofuran

Ts tosyl

### EXAMPLES

### Example 1

### Procedure for the Enantioselective Transfer Hydrogenation of 10-Oxo-10,11-dihydro-dibenzo[b,f]azepine-5-carboxylic acid amide to R(−)-10,11-Dihydro-10-hydroxy-5H-dibenz[b,f]azepine-5-carboxamide

To a mixture of 10-oxo-10,11-dihydro-dibenzo[b,f]azepine-5-carboxylic acid amide (300 mg, 1.189 mmol) and RuCl[(1R,2R)-p-TsNCH(C₆H₅)CH(C₆H₅)NH₂](η⁶-p-cymene, Aldrich, Switzerland) (8.8 mg, 0.0138 mmol) in CH₂Cl₂ (15 ml) is added dropwise a premixed solution of formic acid and NEt₃(5:2, 328 mg:289 mg) at 23° C. and stirred for 10 min. The clear solution is heated to reflux for 16 h. The reaction mixture is cooled to RT, diluted with CH₂Cl₂ (20 ml) and neutralised with aqu. NaHCO₃. After washing with brine the solution is concentrated under reduced pressure. The residue is purified by flash chromatography on silica gel using a 6:1 EtOAc-MeOH mixture as eluent to afford of R(−)-10,11-dihydro-10-hydroxy-5H-dibenzo[b,f]azepine-5-carboxamide (enantiomeric purity (ee)>99% determined by HPLC on Chiracel OD, Retention time: 9.46 min. [α]_{D}^{t}=−195.3° (ethanol). ¹H-NMR (400 MHz, CDCl₃):7.70-7.20 (m, 8 H), 5.30 (br s,1 H), 5.10-4.60 (br s, 2 H), 3.75-3.40 (m, 1 H), 3.20-2.90 (m, 1 H), 2.50 (br s, 2 H). NMR-Datas refer to Lit.: Benes, J et al., J. Med. Chem. 1999, 42, 2582-2587. Molecular weight: 254.291

### Example 2

### Procedure for the Enantioselective Transfer Hydrogenation of 10-Oxo-10,11-dihydro-dibenzo[b,f]azepine-5-carboxylic acid amide to S(+)-10,11-Dihydro-10-hydroxy-5H-dibenz[b,f]azepine-5-carboxamide

To a mixture of 10-oxo-10,11-dihydro-dibenzo[b,f]azepine-5carboxylic acid amide (300 mg, 1.189 mmol) and RuCl[(1S,2S)-p-TsNCH(C₆H₅)CH(C₆H₅)NH₂](η⁶-p-cymene) (11 mg, 0.0173 mmol) in CH₂Cl₂ (15 ml) is added in two portions a premixed solution of formic acid and NEt₃ (5:2, 656 mg:578 mg) at 23° C. and stirred for 10 min. After that formic acid is added (50 μl) and the clear solution is heated to reflux for 16 h. The reaction mixture is cooled to RT, diluted with CH₂Cl₂ (20 ml) and neutralised with aqu. NaHCO₃. After washing with brine the solution is concentrated under reduced pressure. The residue is purified by flash chromatography on silica gel using a 6:1 EtOAc-MeQH mixture as eluent to afford of S(+)-10,11-dihydro-10-hydroxy-5H-dibenzo[b,f]azepine-5-carboxamide (ee>99% by HPLC on Chiracel OD). Retention time: 12.00 min. [α]_{D}^{t}=+196.6° (ethanol). ¹H-NMR (400 MHz, CDCl₃):7.70-7.20 (m, 8 H), 5.30 (br s,1 H), 5.10-4.60 (br s, 2 H), 3.75-3.40 (m, 1 H), 3.20-2.90 (m, 1 H), 2.50 (br s, 2 H). NMR-Datas refer to Lit.: Benes, J et al., J. Med. Chem. 1999, 42, 2582-2587. Molecular weight: 254.291

Alternative production: To a mixture of 10-oxo-10,11-dihydro-dibenzo[b,f]azepine-5-carboxylic acid amide (300 mg, 1.189 mmol) and RuCl[(1S,2S)-p-dansyl-NCH(C₆H₅)CH(C₆H₅)NH₂](η⁶-p-cymene) (8.5 mg, 0.012 mmol) in CH₂Cl₂ (15 ml) is added dropwise a premixed solution of formic acid and NEt₃ (5:2, 328 mg:289 mg) at 23° C. and stirred for 10 min. The clear solution is heated to reflux for 16 h. The reaction mixture is cooled to RT, diluted with CH₂Cl₂ (20 ml) and neutralised with aqu. NaHCO₃. After washing with brine the solution is concentrated under reduced pressure. The residue is purified by flash chromatography on silica gel using a 6:1 EtOAc-MeOH mixture as eluent to afford of S(+)-10,11-dihydro-10-hydroxy-5H-dibenzo[b,f]azepine-5-carboxamide.

### Example 3

### Preparation of RuCl[(1S,2S)-p-dansylNCH(C₆H₅)CH(C₆H₅)NH₂](η⁶-p-cymene)

a) Preparation of (S,S)-5-dimethylamino-naphthalene-1-sulfonic acid (2-amino-1,2diphenyl-ethyl)-amide: To a solution of (S,S)-diphenylethylenediamine (250 mg, 1.2 mmol) and triethylamine (0.5 ml) in THF is added dropwise a solution of dansyl chloride (318 mg, 1.2 mmol) in THF (2 ml) at 0° C. After stirring 16 h at RT the solvent is removed in vacuum and the residue is resolved in methylenchloride (20 ml). The organic solution is washed with NaHCO₃ solution (5 ml), dried over Na₂SO₄ and after filtration the solvent is removed. Flash chromatographie afford (S,S)-5-dimethylamino-naphthalene-1-sulfonic acid (2-amino-1,2-diphenyl-ethyl)-amide as yellow oil which crystallizes by drying in vacuum. M: 445.59. ¹H-NMR (400 MHz, CDCl₃):8.36 (t, J=7.5 Hz, 2 H), 8.17 (dd, J=7.2, 1.2 Hz, 1 H), 7.47 (dd, J=8.8 Hz, 1 H), 7.34 (dd, J=8.5 Hz, 1 H), 7.24-7.16 (m, 4 H), 7.11 (d, J=7.5 Hz, 1 H), 6.99-6.74 (m, 6 H), 4.61 (d, J=8.5 Hz, 1 H), 4.20 (d, J=8.5 Hz, 1 H), 2.80 (s, 6 H).

b) Preparation of RuCl[(1S,2S)-p-dansylNCH(C₆H₅)CH(C₆H₅)NH₂](η⁶-p-cymene): A solution of (S,S)-5-dimethylamino-naphthalene-1-sulfonic acid (2-amino-1,2-diphenyl-ethyl)-amide (80 mg, 0.18 mmol), NEt₃ (36 mg, 0.36 mmol) and [RuCl₂(p-cymene)]₂ (55 mg, 0.09 mmol) in 2-propanol is heated at 80° C. for 1 h. The solvent is removed after that und the dark red residue is washed with water (2 ml). The solid is dried in vacuum and used without any purification. M: 715.34.

### Example 4

### Activity of the Enantiomers of the Compound of Formula I in a Model of Neuropathic Pain in the Guinea-Pig

Neuropathic hyperalgesia is induced by partial ligation of the left sciatic nerve (Seltzer et al, Pain 43, 1990, 205-218; Campbell et al, Neuroscience 87, 1998, 527-532). Briefly, male Dunkin Hartley guinea pigs (200-250 g) are anaesthetized with isoflurane in N₂O:O₂, the left sciatic nerve exposed at mid thigh level through a small incision and ⅓ to ½ of the nerve thickness tightly ligated within a 7.0 silk suture. The wound is closed and the animals are allowed to recover from surgery for 12 to 15 days.

Mechanical hyperalgesia is assessed by measuring paw withdrawal thresholds to an increasing pressure stimulus placed onto the dorsal surface of the paw using an analgesymeter (Ugo-Basile, Milan) with a cut-off of 250 9. Withdrawal are measured on both the ipsilateral (ligated) and contralateral (unligated) paw prior to and then up to 6 h following drug or vehicle administration. Reversal of hyperalgesia at each time point is calculated according to the following formula, which uses the contralateral paw as a reference:

$$\% reversal = \frac{\begin{matrix}ipsilateral threshold postdose - \\ ipsilateral threshold predose\end{matrix}}{\begin{matrix}contralateral threshold predose - \\ ipsilateral threshold predose\end{matrix}} \times 100$$

The enantiomers of the compound of formula I are administered daily in 0.5% methyl-cellulose/water, with Trileptal™ included in each experiment as positive control. Each experiment uses 6 randomly assigned animals per treatment group. Statistical analysis is carried out on withdrawal threshold data comparing test to vehicle.

The R-enantiomer of the compound of formula I produces a dose-related reversal of mechanical hyperalgesia in neuropathic guinea-pigs. A maximum reversal of 73% is observed 1 h following administration with a calculated D₅₀ value of 47 mg/kg. The effect of the R-enantiomer of the compound of formula I is long-lasting with significant activity apparent 6 h following administration. The S-enantiomer of the compound of formula I is markedly less active than the R-enantiomer, producing an apparent maximal reversal of hyperalgesia of 55%. Anti-hyperalgesic activity is observed only with the highest dose tested (100 mg/kg), with lower doses producing no significant effect. Administration of the S-enantiomer is also associated with marked side-effects, principally ataxia and catalepsy.

The obtained results indicate a clear difference in the anti-hyperalgesic activity of the two enantomers of the compound of formula 1, with the R-enantiomer showing greater efficacy and potency than the S-enantiomer, and with a more prolonged duration of action of the R-enantiomer. Moreover, the S-enantiomer produces side-effects at doses that reverses mechanical hyperalgesia, whilst comparatively mild side-effect are observed with the highest dose of the R-enantiomer.

1-8. (canceled)

9. A method for the treatment of neuropathic pain or post-herpetic neuralgia in a subject in need of such treatment, which comprises administering to said subject a therapeutically effective amount of a mixture of the enantiomers of the compound of formula I 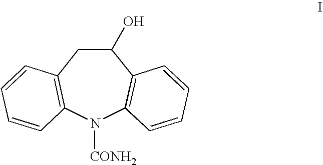
or of pharmaceutically acceptable salts of said enantiomers consisting of at least 55% of the R-enantiomer and not more than 45% of the S-enantiomer.

10. The method of claim 9 wherein the mixture consists of at least 85% of the R-enantiomer and not more than 15% of the S-enantiomer.

11. The method of claim 9 wherein the mixture consists of at least 98% of the R-enantiomer and not more than 2% of the S-enantiomer.

12. A pharmaceutical composition comprising as active agent a mixture of the enantiomers of the compound of formula I 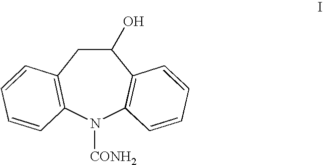
or pharmaceutically acceptable salts of said enantiomers wherein said mixture of enantiomers contains at least 55% of the R-enantiomer and not more than 45% of the S-enantiomer.

13. A package or kit comprising a pharmaceutical composition of claim 12 for administration according to a method for the treatment of neuropathic pain or post-herpetic neuralgia wherein said method provides for a daily dosage of from about 10 mg. to about 3000 mg. of said pharmaceutical composition.
